(19) United States
(12) Patent Application Publication
(10) Pub. No.: US 2005/0131499 A1
(43) Pub. Date: Jun. 16, 2005

(54) Laser device to treat sympathetic and parasympathetic nervous systems

(76) Inventors: Steven C. Shanks, Mesa, AZ (US); Kevin Tucek, Mesa, AZ (US)

Correspondence Address: ETHERTON LAW GROUP, LLC, 5555 E. VAN BUREN STREET, SUITE 100, PHOENIX, AZ, 85008, US

(21) Appl. No.: 10/772,973

(22) Filed: Feb. 4, 2004

Related U.S. Application Data
(63) Continuation-in-part of application No. 09/932,907, filed on Aug. 20, 2001, now Pat. No. 6,746,473.
(60) Provisional application No. 60/273,282, filed on Mar. 2, 2001.

(51) Int. Cl. A61N 5/06

(52) U.S. Cl. 607/89

(57) ABSTRACT

An improved laser device that can simultaneously provide low level laser therapy treatments to the sympathetic and parasympathetic nervous systems. The device enables laser light of different colors, pulse frequencies, beam shapes and spot sizes to be applied externally to a patient's body. The device includes multiple laser sources. In the preferred embodiment, and hand-held wand emits two separate laser beams, one laser beam producing a pulsed line of red laser light and the other producing a pulsed line of green laser light.

### CROSS-REFERENCE TO RELATED APPLICATIONS

This application claims the benefit of co-pending U.S. application Ser. No. 09/932,907 filed Aug. 20, 2001 which claims the benefit of U.S. Provisional Application No. 60/273,282 filed Mar. 2, 2001.

### FIELD OF INVENTION

This invention relates generally to medical devices that employ lasers. More particularly, this invention relates to a single laser light generator device that provides warm and cool radiation.

### BACKGROUND

Low energy laser therapy (LLLT) is used in the treatment of a broad range of conditions. LLLT improves wound healing, reduces edema, and relieves pain of various etiologies, including successful application post-operatively to liposuction to reduce inflammation and pain. It is also used in the treatment and repair of injured muscles and tendons.

LLLT utilizes low level laser energy, that is, the treatment has a dose rate that causes no immediate detectable temperature rise of the treated tissue and no macroscopically visible changes in tissue structure. Consequently, the treated and surrounding tissue is not heated and is not damaged. There are a number of variables in laser therapy including the wavelength of the laser beam, the area impinged by the laser beam, laser energy, pulse frequency, treatment duration and tissue characteristics. The success of each therapy depends on the relationship and combination of these variables. For example, liposuction may be facilitated with one regimen utilizing a given wavelength and treatment duration, whereas pain may be treated with a regimen utilizing a different wavelength and treatment duration, and inflammation a third regimen. Specific devices are known in the art for several types of therapy.

Recent, research has shown that laser light in the cool color range excites the sympathetic subsystem of the autonomic nervous system and that laser light in the warm color range excites the parasympathetic subsystem. Other studies have shown that an imbalance in the sympathetic and parasympathetic systems impairs maximum muscle strength and nerve facilitation. Therefore it would desirable to use LLLT to restore balance between the sympathetic and parasympathetic systems. It would be particularly desirable to provide both such treatments with a single device.

Therefore, an object of this invention is to provide a laser therapy device that treats the sympathetic and parasympathetic systems. It is another object of this invention to provide a single apparatus that can treat these systems with different colors of laser light. It is a further object of this invention to provide a single apparatus that can emit laser light in multiple beam shapes and spot sizes. It is a particular object of this invention to provide a hand-held therapeutic laser device to provide low level laser therapy which can be used to treat the sympathetic and parasympathetic systems.

### SUMMARY OF THE INVENTION

This invention is a laser device that provides low level laser therapy treatments to the sympathetic and parasympathetic nervous systems. The device enables laser light of different colors, pulse frequencies, beam shapes and spot sizes to be applied externally to a patient's body. The device includes multiple laser sources. In the preferred embodiment, a hand-held wand emits two laser beams, one laser beam producing a pulsed line of red laser light and the other producing a pulsed line of green laser light.

### BRIEF DESCRIPTION OF THE DRAWINGS

FIG. 1 is an electrical schematic illustration of a preferred embodiment of the present invention.

FIG. 2 is a schematic view of the optical arrangement of the linear spot shape of the preferred embodiment.

FIG. 3 is a schematic view of the optical arrangement of the circular spot shape of an alternate embodiment.

FIG. 4 is a schematic illustration of application of low-level laser radiation using the preferred embodiment of the present invention.

FIG. 5 is a schematic illustration of application of low-level laser radiation using an alternate embodiment of the present invention.

FIG. 6 is a schematic illustration of application of low-level laser radiation using an alternative embodiment with two wands.

FIG. 7 is a schematic illustration of application of low-level laser radiation using an alternative embodiment of the present invention with a stand-alone source.

### DETAILED DESCRIPTION OF THE INVENTION

FIG. 1 shows the preferred embodiment in which a first laser energy source 11 and a second energy source 12 are connected to a power source 13. The power source preferably provides direct current, such as that provided by a battery, but may instead provide alternating current such as that provided by conventional building current that is then converted to direct current. Separate control means 15, 16 are connected to the laser energy sources 11, 12 respectively and act as on/off switches to control the period of time the laser light is generated. These laser energy sources can be energized independently or simultaneously which, throughout this specification, refers to acts occurring at generally the same time.

Studies have shown that laser light in the warm color range, about 575-780 nm, influences largely the parasympathetic nervous system. Laser light in the cool color range, about 360-575 nm, influences largely the sympathetic nervous system. The root of the parasympathetic nervous system is primarily in the brain, upper cervical, and sacral portion of the spinal cord. The root of the sympathetic nervous system is in the thoracic and lumbar portions of the spinal cord, from level T1 to approximately L2. Thus, laser light can be used for diagnostic and therapeutic modality of between the sympathetic and parasympathetic systems when applied to the appropriate nerve root(s) in the spinal cord.

Laser energy sources are known in the art for use in low-level laser therapy. They include solid state, gas, and semiconductor diode lasers. The preferred embodiment uses semiconductor diode lasers which provide a broad range of wavelengths from mid-infrared to blue. The laser energy sources in the preferred embodiment are two semiconductor laser diodes. The first laser energy source 11 produces light in the red range of the visible spectrum, about 635-700 nm, and preferably 635 nm. The second laser energy source 12 produces light in the green range of the visible spectrum, about 491-575 nm, and preferably 491 nm. Other suitable wavelengths are used for other particular applications. It is advantageous to utilize at least one laser beam in the visible/UV energy spectrum so that the operator can see the laser light as it impinges the patent's body and the area treated can be easily defined. Solid state and tunable semiconductor laser diodes may also be employed to achieve the desired wavelength.

Different therapy regimens require diodes of different wattages. The preferred laser diodes use less than one watt of power each to stimulate nerve roots in the spinal cord. Diodes of various other wattages may also be employed to achieve the desired laser energy for the given regimen.

Control means 21, 22 are connected to the laser energy sources 11, 12, respectively, to form a control circuit that controls the pulse frequency. When there are no pulses, a continuous beam of laser light is generated. Pulse frequencies from 0 to 100,000 Hz may be employed to achieve the desired effect on the patient's tissue. The goal for LLLT regimen is to deliver laser energy to the target tissue utilizing a pulse frequency short enough to sufficiently energize the targeted tissue and avoid thermal damage to adjacent tissue.

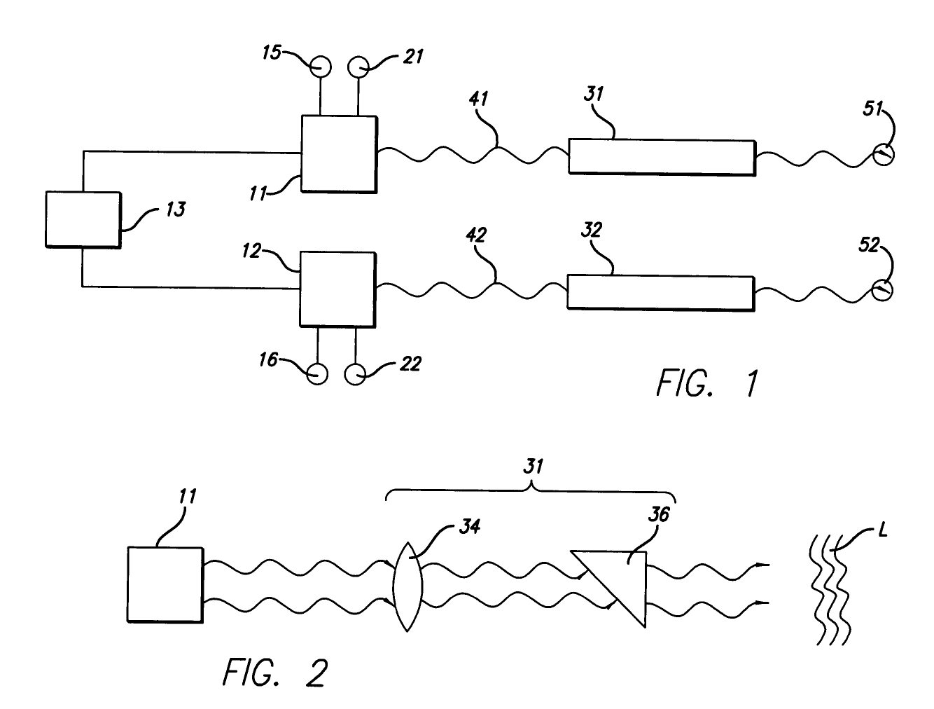

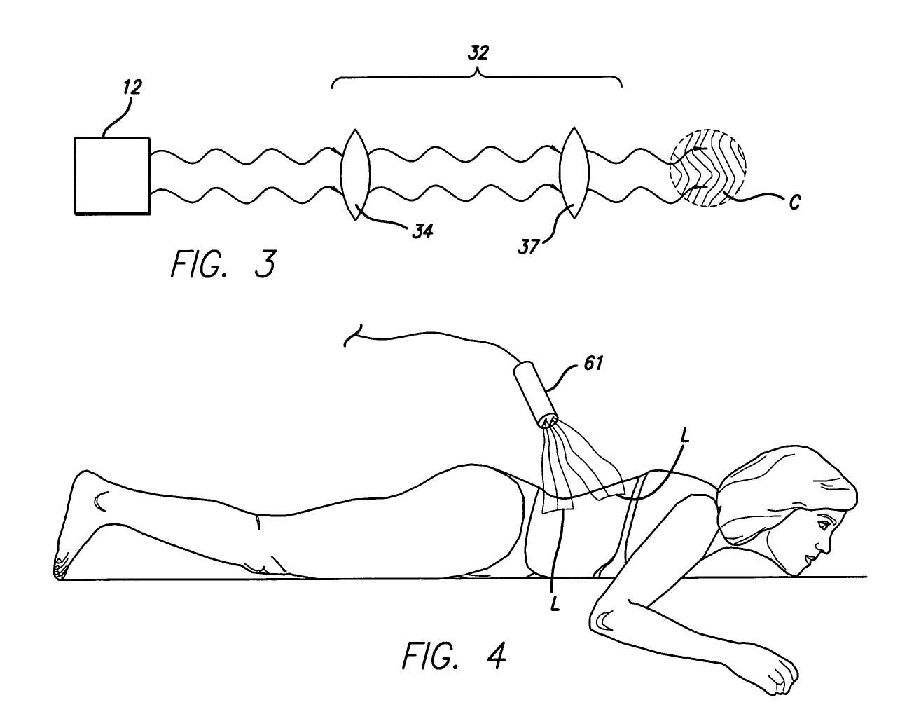

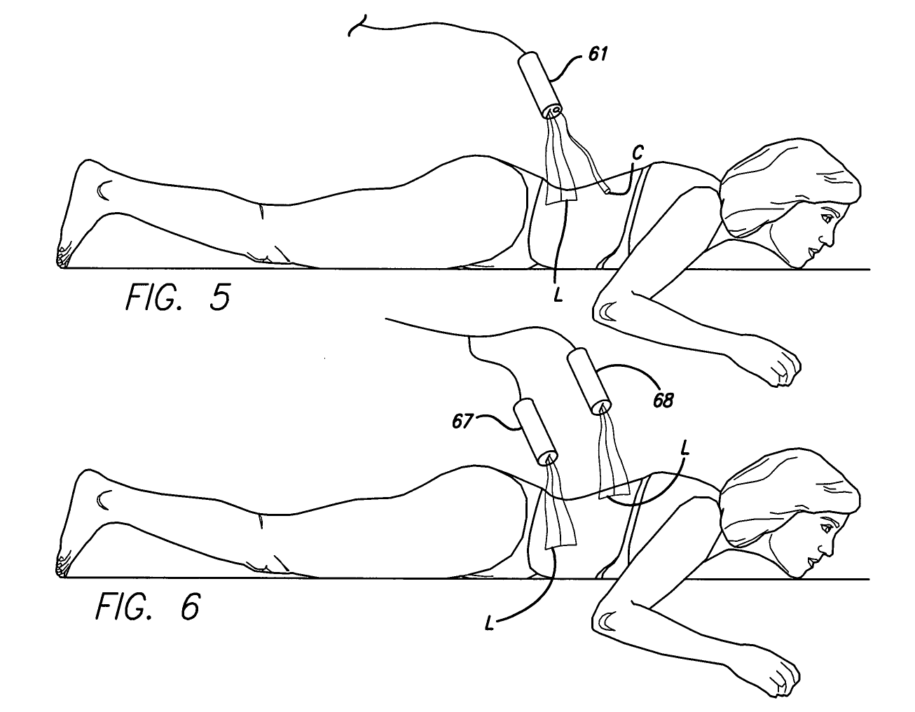

Each laser beam 41, 42 exits the laser and is shone through optical arrangements 31, 32, respectively, that produce beam spots 51, 52 respectively of certain shapes. See FIGS. 2 and 3. The beam spot is the cross-sectional shape and size of the emitted beam as it exits the optical arrangement. For example, a laser beam of circular cross-section creates a circular spot C as the laser light impinges the patient's skin. If the laser light emitted is in the visible range, a circular spot can be seen on the patient's skin of substantially the same diameter as the laser beam emitted from the optics arrangement. In the preferred embodiment, each laser beam passes through an optical arrangement that generates a beam of substantially linear cross-section, resulting in a line of laser light L seen on the patient's skin. See FIG. 4. In an alternative embodiment shown in FIG. 5, one laser provides a linear spot L and a second laser passes through an optical arrangement that generates a beam of circular cross-section, resulting in a circular spot C as seen on the patient's skin.

As shown in FIG. 2 the first optical arrangement 31 of the preferred embodiment includes a collimating lens 34 and a line generating prism 36. The collimating lens 34 and the line generating prism 36 are disposed in serial relation to the laser energy source 11. The collimating lens 34 and the line generating prism 36 receive and transform the generated beam of laser light into the line of laser light L. As an alternative, a suitable electrical or mechanical arrangement could be substituted for the optical arrangement 31.

As shown in FIG. 3 the second optical arrangement 32 of the preferred embodiment includes a collimating lens 34 and a beam spot shaping lens 37. As with the first optical arrangement, the collimating lens 34 and beam spot shaping lens 37 are disposed in serial relation to the laser energy source 12. The collimating lens 34 and beam spot shaping lens 37 receive and transform the generated beam of laser light into a circular beam spot of laser light C. As an alternative, a suitable electrical or mechanical arrangement could be substituted for the optical arrangement 32 to achieve a desired spot shape.

The device may utilize as many lasers and optical arrangements as necessary to obtain the desired emissions and spot shapes. For example, the device may employ two laser diodes each with a collimating lens and beam spot shaping lens, such that two substantially circular spot shapes are achieved. Or, for example, the device may employ two laser diodes each with an optical arrangement such that two substantially linear spot shapes are achieved. Or, in another example, more than two lasers may be used and optical arrangements aligned such that two or more of the laser beams have substantially similar spot shapes and are co-incident where they impinge the patient's skin.

The laser light can be directed to the desired area with a single hand-held wand, multiple hand-held wands, or a standalone device. FIG. 4 shows the preferred embodiment in which the laser light is emitted from a lightweight, hand-held pointer referred to herein as a wand 61. The wand 61 is preferably an elongated hollow tube defining an interior cavity which is shaped to be easily retained in a user's hand. In the preferred embodiment the laser energy sources 11, 12 are mounted in the wand's interior cavity, although the laser energy sources could be remotely located and the laser light conducted by fiber optics to the wand. The wand may take on any shape that enables the laser light to be directed as needed such as tubular, T-shaped, substantially spherical, or rectangular (like a television remote control device).

FIG. 6 shows an alternative embodiment in which the laser light is emitted from multiple wands. This enables the practitioner to apply laser light simultaneously at multiple areas on a patient's body. For the treatment of the sympathetic and parasympathetic systems, the first wand 67 emits green laser light and the second wand 68 emits red laser light. Preferably the beam spots are substantially linear.

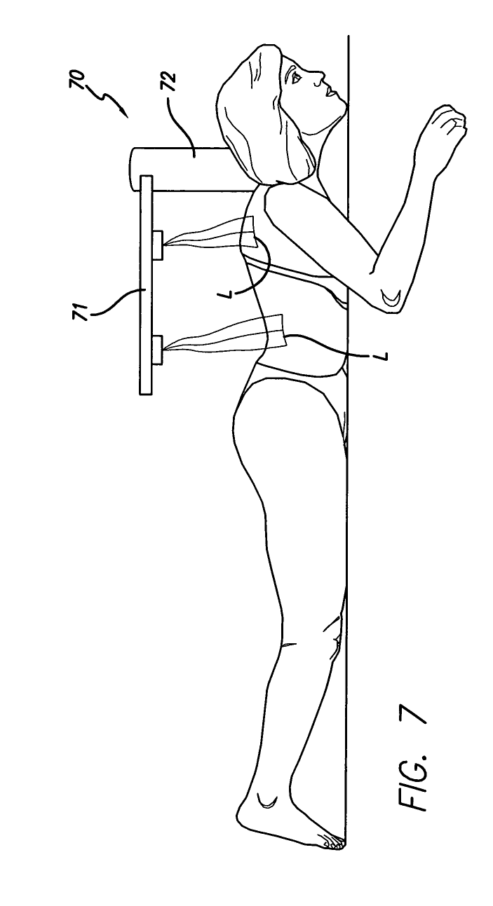

FIG. 7 shows another alternative embodiment in which the laser light is emitted from an arm 71 of a standalone device 70. The standalone device generally comprises the arm 71, a post 72, and a base (not shown), having sufficient weight to prevent the device from tipping. The arm 71 is preferably an elongated hollow tube defining an interior cavity. Laser energy sources 11, 12 are mounted in the arm's interior cavity, although the laser energy sources could be remotely located and the laser light conducted by fiber optics to the arm. The arm 71 is connected to the post 72, preferably in such a way that the arm is freely positionable in the x-, y-, and z-axes. Preferably house current is used as the power source in this alternative embodiment.

While there has been illustrated and described what is at present considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of the appended claims.

1. A laser device comprising:
a) a plurality of laser energy sources for generating a plurality of laser beams in which at least a first laser beam is a cool color and at least a second laser beam is a warm color; and
b) an optical arrangement for receiving at least one laser beam and for transforming at least one laser beam into a desired spot shape.

2. The device according to claim 1 in which the first laser beam is green.

3. The device according to claim 1 in which the first laser beam is red.

4. The device according to claim 1 in which the desired spot shape is substantially linear.

5. The device according to claim 1 in which each of the plurality of laser beams has a desired spot shape that is substantially linear.

6. A device according to claim 1 wherein at least two of the laser beams are emitted simultaneously.

7. A laser device comprising:
a) a plurality of laser energy sources for generating a plurality of laser beams in which at least a first laser beam is a cool color and at least a second laser beam is a warm color;
b) a wand from which the laser beams emit, the wand being capable of being retained in a hand of a user and freely moved relative to the surface of the skin of a patient; and
c) an optical arrangement attached to the wand for receiving the laser beams and for transforming each of the laser beams into a desired spot shape.

8. The device according to claim 7 in which the first laser beam is green.

9. The device according to claim 7 in which the first laser beam is red.

10. A device according to claim 7 wherein at least two of the laser beams are emitted simultaneously.

11. A device according to claim 7 further comprising a controller for independently controlling the generation of laser energy by each of the plurality of laser energy sources.

12. A device according to claim 7 wherein each of the laser energy sources is less than one watt.

13. A device according to claim 7 wherein at least one of the laser energy sources is a semiconductor diode.

14. A device according to claim 7 wherein at least one of the spot shapes is substantially linear.

15. A device according to claim 7 further comprising a first laser beam having a first spot shape and a second laser beam having a second spot shape wherein the first spot shape is substantially linear and the second spot shape is circular.

16. A device according to claim 7 further comprising a control circuit for controlling the pulse frequency of each laser beam.

17. A device according to claim 16 wherein the pulse frequency of at least one of the laser beams is such that the laser light emitted is substantially continuous.

18. A device according to claim 16 further comprising a first laser beam having a first pulse frequency and a second laser beam having a second pulse frequency wherein the first pulse frequency is such that the laser light emitted is substantially continuous and the second pulse frequency is not zero.

19. A device according to claim 16 wherein the pulse frequency of the second laser beam is less than 100,000 Hz.

20. A laser device comprising:
a) a plurality of laser energy sources for generating a plurality of laser beams in which at least a first laser beam is a cool color and at least a second laser beam is a warm color;
b) an arm from which the laser beams emit, the arm being capable of being freely positionable in the x-, y-, and z-axes; and
c) an optical arrangement attached to the arm for receiving the laser beams and for transforming each of the laser beams into a desired spot shape.

21. The device according to claim 20 in which the first laser beam is green.

22. The device according to claim 20 in which the first laser beam is red.

23. A device according to claim 20 wherein at least two of the laser beams are emitted simultaneously.

24. A device according to claim 20 further comprising a controller for independently controlling the generation of laser energy by each of the plurality of laser energy sources.

25. A device according to claim 20 wherein each of the laser energy sources is less than one watt.

26. A device according to claim 20 wherein at least one of the laser energy sources is a semiconductor diode.

27. A device according to claim 20 wherein at least one of the spot shapes is substantially linear.

28. A device according to claim 20 further comprising a first laser beam having a first spot shape and a second laser beam having a second spot shape wherein the first spot shape is substantially linear and the second spot shape is circular.

29. A device according to claim 20 further comprising a control circuit for controlling the pulse frequency of each laser beam.

30. A device according to claim 20 wherein the pulse frequency of at least one of the laser beams is such that the laser light emitted is substantially continuous.

31. A device according to claim 20 further comprising a first laser beam having a first pulse frequency and a second laser beam having a second pulse frequency wherein the first pulse frequency is such that the laser light emitted is substantially continuous and the second pulse frequency is not zero.

32. A device according to claim 20 wherein the pulse frequency of the second laser beam is less than 100,000 Hz.

33. A device for treating the sympathetic and parasympathetic nervous systems comprising:
a) a first laser energy source that emits a green laser beam;
b) a second laser energy source that emits a red laser beam;
c) a wand from which the laser beams emit, the wand being capable of being retained in a hand of a user and freely moved relative to the surface of the skin of a patient; and
d) an optical arrangement attached to the wand for receiving the laser beams and for transforming each of the laser beams into a substantially linear spot shape.
